(19) United States
(12) Patent Application Publication
(10) Pub. No.: US 2009/0171098 A1
(43) Pub. Date: Jul. 2, 2009

(54) Heteroaryl Salts and Methods For Producing and Using the Same

(75) Inventors: Jason E. Bara, Denver, CO (US); Evan S. Hatakeyama, Boulder, CO (US); Richard D. Noble, Boulder, CO (US); Douglas L. Gin, Longmont, CO (US)

Correspondence Address: Don D. Cha, 547 Buena Vista Road, Golden, CO, 80401, US

(73) Assignee: THE REGENTS OF THE UNIVERSITY OF COLORADO, Denver, CO (US)

(21) Appl. No.: 12/333,052

(22) Filed: Dec. 11, 2008

Related U.S. Application Data
(60) Provisional application No. 61/012,893, filed on Dec. 11, 2007.

(51) Int. Cl. C07D 233/58

(52) U.S. Cl. 548/335.1

(57) ABSTRACT

The invention provides heteroaryl salts and methods for producing the same. In particular, the invention provides heteroaryl salts of the formula:

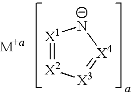

and methods for producing the same, where M, a, X¹, X², X³, and X⁴ are those defined herein.

### CROSS-REFERENCE TO RELATED APPLICATIONS

This application claims the priority benefit of U.S. Provisional Application No. 61/012,893, filed Dec. 11, 2007, which is incorporated herein by reference in its entirety.

### STATEMENT REGARDING FEDERALLY FUNDED RESEARCH

The U.S. Government has a paid-up license in this invention and the right in limited circumstances to require the patent owner to license others on reasonable terms as provided for by the terms of Grant No. DMR-0552399 awarded by the National Science Foundation.

### FIELD OF THE INVENTION

The present invention relates to heteroaryl salts and methods for producing the same.

### BACKGROUND OF THE INVENTION

Salts heteroaryl compounds are convenient starting materials in the synthesis of substituted heteroaryl compounds such as N-substituted imidazoles. Substituted heteroaryl compounds are present in a wide variety of useful compounds including biologically and medicinally useful compounds, such as antibacterial and antifungal agents, and surfactants. In most instances, heteroaryl salts, for example, sodium imidazolate or imidazole sodium salt (“ISS”), are generated in situ for use as a nucleophile. Often these salts are generated in situ in the same reaction vessel that is used in a subsequent reaction. A common method for producing heteroaryl salts, such as ISS, from a heteroaryl compound containing a relatively acidic N—H group is to deprotonate the N—H bond on the heteroaryl compound with a strong base such as sodium hydride (NaH) in an anhydrous organic solvent such as tetrahydrofuran (THF) or N,N-dimethylformamide (DMF). Some heteroaryl salts, such as ISS, are insoluble in these solvents and precipitate as a solid. Often an electrophilic compound, e.g. alkyl halide, is then added to the reaction mixture, where it dissolves in the solvent. Heating and often vigorous stirring allow reaction between solid heteroaryl salt and the electrophilic compound to produce the substituted heteroaryl compound, e.g., an N-substituted imidazole.

Thus, conventional methods for producing large quantities of heteroaryl salts, such as ISS, are rather expensive processes because they require a large volume of anhydrous solvents to maintain fluidity in the reaction vessel, otherwise precipitation of heteroaryl salts (e.g., ISS) limits reaction mixing, impeding production of more heteroaryl salts. In addition, while sodium hydride is rather inexpensive and can be used for the deprotonation of many heteroaryl compounds including imidazole, its use requires concerns and measures to ensure safe handling and storage, thereby adding labor and cost to the overall process. Moreover, conventional processes often require conducting the reaction under an inert atmosphere further increasing the cost. When purification of heteroaryl salt is needed, such processes often require washing with more organic solvent and separation from any excess sodium hydride. Thus, generation of heteroaryl salts in situ not only increases cost and labor, it also reduces the number and/or the amount of various substituted heteroaryl compounds produced during a given time period.

Therefore, there is a need for a method for generating a large quantity of heteroaryl salts relatively inexpensively.

### SUMMARY OF THE INVENTION

Some aspects of the invention provide isolated heteroaryl compounds of Formula I and methods for producing the same. Typically, the heteroaryl compounds of the invention are at least about 95% pure and are of the formula:

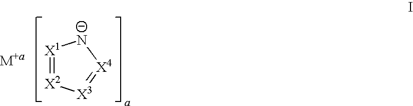

wherein
a is an oxidation state of M;
M is a metal, or R^{1a}R^{2a}R^{3a}N⁺, wherein each of R^{1a}, R^{2a}, R^{3a}, and R^{4a} is independently hydrogen or alkyl; and
each of X¹, X², X³, and X⁴ is independently N or CR⁵;
wherein
   each R⁵ is independently hydrogen, halide, alkyl, heteroalkyl, aryl, aralkyl, cycloalkyl, (cycloalkyl)alkyl, heteroaryl, heteroaralkyl, heterocyclyl, or (heterocyclyl)alkyl;
   or two adjacent R⁵'s along with the carbon atoms to which they are attached to form an optionally substituted aryl, heteroaryl, cyclycl, or heterocyclyl.

Other aspects of the invention provide methods for producing room-temperature ionic liquids having an imidazole core structure. In particular, some embodiments of the invention provide methods for producing a room-temperature ionic liquid (RTIL) compound of the formula:

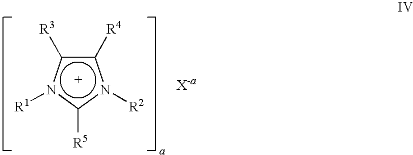

said method comprising
(i) reacting a compound of the formula:

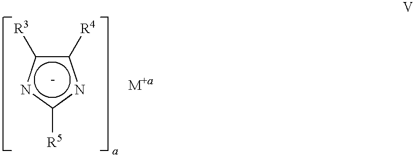

with a first reagent of the formula R¹-Z¹ under conditions sufficient to produce a mono-nitrogen substituted imidazole compound of the formula:

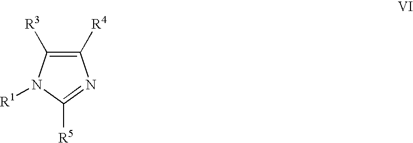

(ii) reacting compound of Formula VI with a second reagent of the formula R²-Z² under conditions sufficient to produce a RTIL compound of the formula:

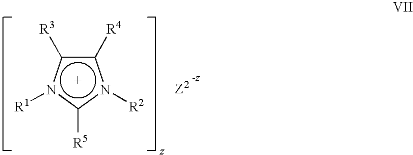

and
(iii) when Z² is different from X, then reacting compound of Formula VII with M¹ₐXₘ to produce the RTIL compound of Formula IV
   wherein
a is an oxidation state of X;
m is an oxidation state of M¹;
z is an oxidation state of Z²;
X is a counter anion; and
each of R¹ and R² is independently alkyl, heteroalkyl, cycloalkyl, haloalkyl, silyl, siloxyl, aryl, alkenyl, or alkynyl;
each of R³, R⁴, and R⁵ is independently hydrogen, alkyl, cycloalkyl, heteroalkyl, haloalkyl, silyl, siloxyl, aryl, alkenyl, or alkynyl;
each of M and M¹ is independently a metal, or R^{1a}R^{2a}R^{3a}R^{4a}N⁺, wherein each of R^{1a}, R^{2a}, R^{3a}, and R^{4a} is independently hydrogen or alkyl; and
each of Z¹ and Z² is independently a leaving group.

### DETAILED DESCRIPTION OF THE INVENTION

### Definitions

“Alkyl” refers to a saturated linear monovalent hydrocarbon moiety of one to twelve, preferably one to six, carbon atoms or a saturated branched monovalent hydrocarbon moiety of three to twelve, preferably three to six, carbon atoms. Exemplary alkyl group include, but are not limited to, methyl, ethyl, n-propyl, 2-propyl, tert-butyl, pentyl, and the like.

“Alkylene” refers to a saturated linear saturated divalent hydrocarbon moiety of one to twelve, preferably one to six, carbon atoms or a branched saturated divalent hydrocarbon moiety of three to twelve, preferably three to six, carbon atoms. Exemplary alkylene groups include, but are not limited to, methylene, ethylene, propylene, butylene, pentylene, and the like.

“Aryl” refers to a monovalent mono-, bi- or tricyclic aromatic hydrocarbon moiety of 6 to 15 ring atoms which is optionally substituted with one or more, preferably one, two, or three substituents within the ring structure. When two or more substituents are present in an aryl group, each substituent is independently selected.

“Aralkyl” refers to a moiety of the formula —R^{b}R^{c} where R^{b} is an alkylene group and R^{c} is an aryl group as defined herein. Exemplary aralkyl groups include, but are not limited to, benzyl, phenylethyl, 3-(3-chlorophenyl)-2-methylpentyl, and the like.

“Cycloalkyl” refers to a non-aromatic, saturated or unsaturated, monovalent mono- or bicyclic hydrocarbon moiety of three to ten ring carbons. The cycloalkyl can be optionally substituted with one or more, often one, two, or three, substituents within the ring structure. When two or more substituents are present in a cycloalkyl group, each substituent is independently selected.

“(Cycloalkyl)alkyl” refers to a moiety of the formula —R^{d}R^{e} where R^{d} is an alkylene group and R^{e} is a cycloalkyl group as defined herein. Exemplary (cycloalkyl)alkyl groups include, but are not limited to, cyclopropylmethyl, cyclohexylpropyl, 3-cyclohexyl-2-methylpropyl, and the like.

The terms “halo,” “halogen” and “halide” are used interchangeably herein and refer to fluoro, chloro, bromo, or iodo.

“Haloalkyl” refers to an alkyl group as defined herein in which one or more hydrogen atom is replaced by same or different halides. The term “haloalkyl” also includes perhalogenated alkyl groups in which all alkyl hydrogen atoms are replaced by halogen atoms. Exemplary haloalkyl groups include, but are not limited to, —CH₂Cl, —CF₃, —CH₂CF₃, —CH₂CCl₃, and the like. Often haloalkyl is fluoroalkyl.

As used herein, the term “heteroalkyl” means a branched or unbranched, cyclic or acyclic saturated alkyl moiety containing carbon, hydrogen and one or more heteroatoms in place of a carbon atom, or optionally one or more heteroatom-containing substituents independently selected from ═O, —OR^{a}, —C(O)R^{a}, —NR^{b}R^{c}, —C(O)NR^{b}R^{c}, —CN, and —S(O)ₙR^{d};

where
n is an integer from 0 to 2;
R^{a} is hydrogen, alkyl, haloalkyl, cycloalkyl, cycloalkylalkyl, heterocyclyl, heterocyclylalkyl, aryl, aralkyl, heteroaryl, heteroaralkyl, or acyl;
R^{b} is hydrogen, alkyl, haloalkyl, cycloalkyl, cycloalkylalkyl, heterocyclyl, heterocyclylalkyl, aryl, aralkyl, heteroaryl, heteroaralkyl, or acyl;
R^{c} is hydrogen, alkyl, haloalkyl, cycloalkyl, cycloalkylalkyl, heterocyclyl, heterocyclylalkyl, aryl, aralkyl, acyl, alkylsulfonyl, carboxamido, or mono- or di-alkylcarbomoyl;
or R^{b} and R^{c} can be combined together with the nitrogen to which each is attached to form a four-, five-, six- or seven-membered heterocyclic ring (e.g., a pyrrolidinyl, piperidinyl or morpholinyl ring); and
R^{d} is hydrogen (provided that n is 0), alkyl, haloalkyl, cycloalkyl, cycloalkylalkyl, heterocyclyl, heterocyclylalkyl, aryl, aralkyl, heteroaryl, heteroaralkyl, acyl, amino, monsubstituted amino, disubstituted amino, or hydroxyalkyl.
   Representative examples of R^{d} include, for example, 2-methoxyethyl, benzyloxymethyl, thiophen-2-ylthiomethyl, 2-hydroxyethyl, and 2,3-dihydroxypropyl. Often heteroalkyl is hydroxyalkyl, aminoalkyl, or nitrile alkyl (i.e., —R^{a}CN, where R^{a} is alkylene). More often heteroalkyl is hydroxyalkyl or nitrile alkyl.

The term “heteroaryl” means a monovalent monocyclic or bicyclic aromatic moiety of 5 to 12 ring atoms containing one, two, or three ring heteroatoms selected from N, O, or S, the remaining ring atoms being C. The heteroaryl ring is optionally substituted independently with one or more substituents, typically one or two substituents. More specifically the term heteroaryl includes, but is not limited to, pyridyl, furanyl, thiophenyl, thiazolyl, isothiazolyl, triazolyl, imidazolyl, isoxazolyl, pyrrolyl, pyrazolyl, pyrimidinyl, benzofuranyl, isobenzofuranyl, benzothiazolyl, benzoisothiazolyl, benzotriazolyl, indolyl, isoindolyl, benzoxazolyl, quinolyl, isoquinolyl, benzimidazolyl, benzisoxazolyl, benzothiophenyl, dibenzofuran, and benzodiazepin-2-one-5-yl, and the like.

“Heteroaralkyl” means a moiety —R^{a}R^{b} where R^{a} is an alkylene group and R^{b} is a heteroaryl group as defined above, e.g., pyridin-3-ylmethyl, 3-(benzofuran-2-yl)propyl, and the like.

“Heterocyclyl” means a non-aromatic monocyclic moiety of three to eight ring atoms in which one or two ring atoms are heteroatoms selected from N, O, or S(O)ₙ (where n is an integer from 0 to 2), the remaining ring atoms being C, where one or two C atoms can optionally be a carbonyl group. The heterocyclyl ring can be optionally substituted independently with one or more, preferably one, two, or three, substituents. When two or more substituents are present in a heterocyclyl group, each substituent is independently selected.

“(Heterocyclyl)alkyl” means a moiety —R^{a}R^{b} where R^{a} is an alkylene group and R^{b} is a heterocyclyl group as defined above, e.g., tetrahydropyran-2-ylmethyl, 4-methylpiperazin-1-ylethyl, 2-, or 3-piperidinylmethyl, and the like.

“Leaving group” has the meaning conventionally associated with it in synthetic organic chemistry, i.e., an atom or a group capable of being displaced by a nucleophile and includes halo (such as chloro, bromo, and iodo), alkanesulfonyloxy, arenesulfonyloxy, alkylcarbonyloxy (e.g., acetoxy), arylcarbonyloxy, mesyloxy, tosyloxy, trifluoromethanesulfonyloxy, aryloxy (e.g., 2,4-dinitrophenoxy), methoxy, N,O-dimethylhydroxylamino, and the like.

“Protecting group” refers to a moiety, except alkyl groups, that when attached to a reactive group in a molecule masks, reduces or prevents that reactivity. Examples of protecting groups can be found in T. W. Greene and P. G. M. Wuts, Protective Groups in Organic Synthesis, 3^{rd} edition, John Wiley & Sons, New York, 1999, and Harrison and Harrison et al., Compendium of Synthetic Organic Methods, Vols. 1-8 (John Wiley and Sons, 1971-1996), which are incorporated herein by reference in their entirety. Representative hydroxy protecting groups include acyl groups, benzyl and trityl ethers, tetrahydropyranyl ethers, trialkylsilyl ethers and allyl ethers. Representative amino protecting groups include, formyl, acetyl, trifluoroacetyl, benzyl, benzyloxycarbonyl (CBZ), tert-butoxycarbonyl (Boc), trimethyl silyl (TMS), 2-trimethylsilyl-ethanesulfonyl (SES), trityl and substituted trityl groups, allyloxycarbonyl, 9-fluorenylmethyloxycarbonyl (FMOC), nitro-veratryloxycarbonyl (NVOC), and the like.

“Corresponding protecting group” means an appropriate protecting group corresponding to the heteroatom (i.e., N, O, P or S) to which it is attached.

As used herein, the term “treating”, “contacting” or “reacting” refers to adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

As used herein, the terms “those defined above” and “those defined herein” when referring to a variable incorporates by reference the broad definition of the variable as well as preferred, more preferred and most preferred definitions, if any.

### Heteroaryl Salts

Some aspects of the invention provide heteroaryl salts and methods for producing the same. In one particular aspect, the invention provides a heteroaryl salt of the formula:

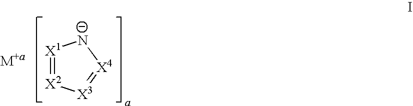

where
a is an oxidation state of M;
M is a metal, or R^{1a}R^{2a}R^{3a}R^{4a}N⁺, where each of R^{1a}, R^{2a}, R^{3a}, and R^{4a} is independently hydrogen or alkyl; and
each of X¹, X², X³, and X⁴ is independently N or CR⁵;
where
   each R⁵ is independently hydrogen, halide, alkyl, heteroalkyl, aryl, aralkyl, cycloalkyl, (cycloalkyl)alkyl, heteroaryl, heteroaralkyl, heterocyclyl, or (heterocyclyl)alkyl;
   or two adjacent R⁵'s along with the carbon atoms to which they are attached to form an optionally substituted aryl, heteroaryl, cyclycl, or heterocyclyl.

The purity of heteroaryl salts of the invention is at least about 90%, often at least about 95%, and more often at least about 98%. Some of the specific heteroaryl salts of the invention include, but are not limited to, imidazole salts, benzimidazole salts, and triazole salts.

Typically, M is an alkali metal, an alkaline earth metal, or a transition metal. Alkali metals are those in Group I of the periodic table. Exemplary alkali metals include, but are not limited to, sodium, potassium, lithium, etc. Alkaline earth metals are those in Group II of the periodic table. Exemplary alkaline earth metals include, but are not limited to, calcium magnesium, etc. Exemplary transition metals include, but are not limited to, chromium, manganese, iron, copper, nickel, cobalt, zinc, silver, gold, etc. Often M is an alkali metal, more often M is sodium, potassium, or lithium, and most often M is sodium.

In other embodiments, M can be an ammonium moiety such as ammonium, tetrahydrocarbyl ammonium (e.g., tetrabutyl ammonium and tetraethyl ammonium), trihydrocarbyl ammonium (e.g., triethyl ammonium, diisopropyl ethyl ammonium and trimethyl ammonium), dihydrocarbyl ammonium, nitrogen heteroaromatic cation (such as 2,6-lutidinium, methyl 2,6-lutidinium, methylpyridinium and pyridinium), or imminium cation. M can also be a phosphonium moiety including tetraalkylphosphonium, tetraaryl phosphonium and phosphonium ions containing a mixture of alkyl and aryl groups; sulfonium moieties such as sulfonium ions containing alkyl, aryl or mixtures thereof; and other suitable cations such as thallium. “Hydrocarbyl” refers to a moiety having at least one carbon atom. Such moieties include aryl, alkyl, alkenyl, alkynyl and a combination of two or more thereof. Moreover, hydrocarbyl can be a straight chain, a branched chain, or a cyclic system. Hydrocarbyl can also be substituted with other non hydrogen or carbon atoms such as halide, oxygen, sulfur, or nitrogen.

Use of commodity bases such as hydroxides, alkoxides, etc. in methods of the present invention, typically under solvent-free (or nearly solvent free) conditions, enables heteroaryls, such as imidazolates, and/or subsequent products be synthesized for a fraction of the cost of a similar reaction using bases such as sodium hydride with anhydrous, volatile organic solvents (such as THF). Methods of the invention also eliminate by-products such as a hydrogen gas that is typically associated with acid-base reactions involving NaH. Products and by-products can be separated more cleanly, and emissions of volatile solvents can be reduced or eliminated.

The variable a is typically 1 or 2. Often a is 1.

In one particular embodiment, the heteroaryl salt is of the formula:

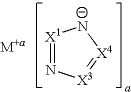

In some embodiments, X¹, X³ and X⁴ are CR⁵. Within these embodiments, often each R⁵ is independently H, halide, or alkyl, and more often R⁵ is H.

The heteroaryl salts of the invention can exist in unsolvated forms as well as solvated forms, including hydrated forms. In general, the solvated forms are equivalent to nonsolvated forms and are intended to be encompassed within the scope of the invention. It should be appreciated, however, one skilled in the art can easily remove or substantially eliminate the solvent from the heteroaryl salts of the invention by drying the salts using methods known to one skilled in the art. In this manner, heteroaryl salts having less than about 5% solvent, typically less than 3%, and more typically less than 1% solvent can be prepared.

Other aspects of the invention provide methods for producing the heteroaryl salt of Formula I. Methods for producing a heteroaryl compound of Formula I comprise reacting a compound of the formula:

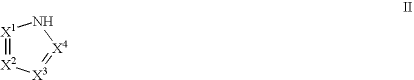

with a hydroxide compound of the formula:

M(OR)ₐ III

under conditions sufficient to produce the heteroaryl compound of Formula I,
where
a, X¹, X², X³, and X⁴ are those defined herein; and
each R is independently hydrogen, alkyl, cycloalkyl, or phenyl; typically, each R is independently hydrogen or alkyl, and often R is hydrogen.

In some embodiments, the reaction is carried out under a reduced pressure. Without being bound by any theory, reduced pressure aids in removal of ROH that is generated in the reaction. In some instances, removal of ROH from the reaction mixture increases the yield of heteroaryl salt of Formula I in accordance with the LeChatelier's principle. When reduced pressure is utilized, typical reaction pressure is about 0.50 atm (7.5 psia) or less, often about 0.25 atm (3.8 psia) or less, and more often about 0.10 atm (1.5 psia) or less.

In some embodiments, the reaction is carried out at an elevated temperature. As expected, raising the temperature often speeds up the reaction and/or increases the product yield. Typically, the reaction is carried out at a temperature of at least about 90° C., often at least about 100° C., and more often at least about 110° C. In some cases, the reaction is carried out at or near the boiling point of the solvent used. Often the reaction is carried out substantially in the absence of any solvent. In such instances, the reaction temperature is at or above the melting temperature of compound of Formula II or III. It should be appreciated, however, that the reaction temperature is not limited those disclosed herein. A wide range of reaction temperature can be used. Generally, a relatively high reaction temperature is used to carry out the reaction within a reasonable reaction time. Typically, the reaction temperature depends on melting point of the starting material(s), such as compound of Formula II. If compound of Formula II does not melt at temperature below 110° C., water or other solvent can be added to facilitate the reaction.

Yet in other embodiments, the reaction is carried out in an aqueous solution. In general, however, the solvent for the reaction is often determined by the identity of compound of Formula III, M(OR)ₐ, used. Typically, the solvent is HOR, for example, when compound of Formula III is sodium methoxide, the solvent used is methanol.

Still in other embodiments, the reaction is carried out in substantially a solvent free condition. In these embodiments, compound of Formula II and compound of Formula III are combined without adding a solvent to the reaction mixture and allowed to react to produce heteroaryl salts of Formula I. Because compound of Formula III is typically a solid, in these embodiments, typically the reaction temperature is raised, if possible, to at least partially melt compound of Formula III.

Other aspects of the invention provide methods for producing room-temperature ionic liquid (RTIL) compounds. In particular, some aspects of the present invention provide methods for producing RTIL compounds having an imidazole core structure. RTIL's are typically salts that are liquid over a wide temperature range including room temperature. Variations in cations and anions can produce a wide variety of ionic liquids including chiral, fluorinated, and antimicrobial RTIL'S. Thus, a large number of possibilities allow for fine-tuning the RTIL properties for specific applications. Typically, RTIL's include bulky and asymmetric organic cations. A wide range of anions are employed, from simple halides, which generally inflect high melting points, to inorganic anions such as tetrafluoroborate and hexafluorophosphate and to large organic anions like bistriflimide, triflate or tosylate. RTIL's have been used in a wide variety of applications including, but not limited to, as a medium for the formation and stabilization of catalytically active transition metal nanoparticles. In some instances, RTIL's can be made that incorporate coordinating groups. Some embodiments of the invention provide methods for producing RTIL compounds of the formula:

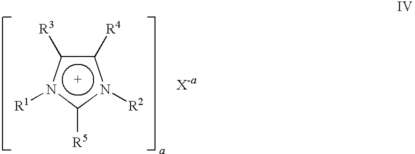

Such methods generally comprise:
(i) reacting a compound of the formula:

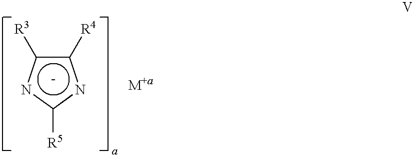

with a first reagent of the formula R¹-Z¹ under conditions sufficient to produce a mono-nitrogen substituted imidazole compound of the formula:

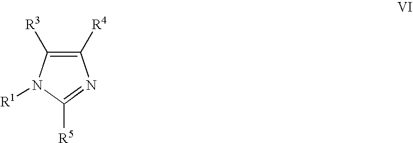

(ii) reacting compound of Formula VI with a second reagent of the formula R²-Z² under conditions sufficient to produce a RTIL compound of the formula:

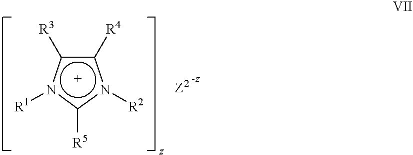

and
(iii) when Z² is different from X, then reacting compound of Formula VII with M¹ₐXₘ to produce the RTIL compound of Formula IV
   where
a is an oxidation state of X;
m is an oxidation state of M¹;
z is an oxidation state of Z²;
X is a counter anion; and
each of R¹ and R² is independently alkyl, heteroalkyl, cycloalkyl, haloalkyl, silyl, siloxyl, aryl, alkenyl, or alkynyl;
each of R³, R⁴, and R⁵ is independently hydrogen, alkyl, cycloalkyl, heteroalkyl, haloalkyl, silyl, siloxyl, aryl, alkenyl, or alkynyl;
each of M and M¹ is independently a metal, or R^{1a}R^{2a}R^{3a}R^{4a}N⁺, wherein each of R^{1a}, R^{2a}, R^{3a}, and R^{4a} is independently hydrogen or alkyl; and
each of Z¹ and Z² is independently a leaving group.

In some embodiments, R¹ is alkyl, haloalkyl, or heteroalkyl. Typically R¹ is alkyl, fluoroalkyl, hydroxyalkyl, or nitrile alkyl.

In other embodiments, R² is alkyl, haloalkyl, or heteroalkyl. Typically R² is alkyl, fluoroalkyl, hydroxyalkyl, or nitrile alkyl.

Still in other embodiments, R³, R⁴, and R⁵ are hydrogen.

There are a wide variety of leaving groups Z¹ and Z² that are suitable for methods of the invention. Typically, any known leaving group in a nucleophilic substitution reaction can be used. Exemplary leaving groups include halides (in particular chloride, bromide, and iodide), tosylates, mesylates, triflates, etc.

Methods of the invention also include producing bi-heteroaryl compounds, such as bis-imidazolium compounds. For example, by using Z¹-R¹-Z¹ in place of R¹-Z¹ as the first reagent. In such instances, each Z¹ can be different leaving groups or can be the same leaving group. Typically, when each Z¹ is different leaving group, one can take advantage of the different reactivity to produce asymmetric bi-heteroaryl compounds. When both Z¹ groups are the same, one can take advantage of the stoichiometric amount of the reagents to produce asymmetric bi-heteroaryl compounds. For example, by using less than one molar equivalent, typically about one-half molar equivalents or less, of compound of Formula V relative to the amount of Z¹-R¹-Z¹ and subsequently reacting the product with a different compound of Formula V, one can produce an asymmetric bi-heteroaryl compound as the major product. In this manner, a wide variety of bis-heteroaryl compounds can be produced using methods of the invention.

It should be appreciated that certain combination of various variables form other embodiments. For example, in some embodiments, R¹ and R² are independently alkyl or hydroxyalkyl and R³, R⁴, and R⁵ are hydrogen. In this manner, a wide variety of embodiments are encompassed within the scope of the present invention.

Some aspects of the invention provide compositions comprising an ionic liquid (IL) heteroaryl compounds of the invention and an amine compound. Compositions of the invention can also include a solvent. When present, the solvent is typically an organic solvent, water, or a combination thereof. Exemplary organic solvents that can be used with compositions and methods of the invention include, but are not limited to, methanol, ethanol, propanol, glycols, acetonitrile, dimethyl sulfoxide, sulfolane, dimethylformamide, acetone, dichloromethane, chloroform, tetrahydrofuran, ethyl actetate, 2-butanone, toluene, as well as other organic solvents known to one skilled in the art.

In some embodiments, the ionic liquid is an imidazolium-based IL, typically an imidazolium-based RTIL. RTILs can be synthesized as custom or “task-specific” compounds with functional groups that enhance physical properties, provide improved interaction with solutes, or are themselves chemically reactive. Multiple points are available for tailoring within the imidazolium-based IL, presenting a seemingly infinite number of opportunities to design ILs matched to individual solutes of interest. Furthermore, many imidazolium-based ILs are miscible with one another or with other solvents; thus, mixtures of ILs serve to multiply the possibilities for creating a desired solvent for any particular application. Separations involving liquids or gases are just one area where the design of selective ILs is of great utility and interest.

The compositions of the present invention include an amine compound. In some embodiments, the amine compound is a heteroalkylamine compound. Within these embodiments, in some instances, the amine compound is an alkanolamine compound. Typically, alkanolamine compound comprises a primary amine group. In other instances, the alkanolamine compound comprises a primary hydroxyl group. Typically, the alkanolamine compound comprises C₂-C₁₀ alkyl chain and often C₂-C₆ alkyl chain. However, it should be appreciated the length of the alkyl chain is not limited to these specific ranges and examples given herein. The length of the alkyl chain can vary in order to achieve a particular property desired.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

Imidazole, sodium salt (i.e., sodium imidazolate, sodium imidazolide, imidazole sodium derivative, etc.) and other metal salts of imidazole or other heteroaryls, are convenient starting materials in the synthesis of various N-substituted heteroaryl compounds. N-Substituted imidazoles are important chemicals that are found in many applications. A typical ISS synthesis is outlined in Scheme I below.

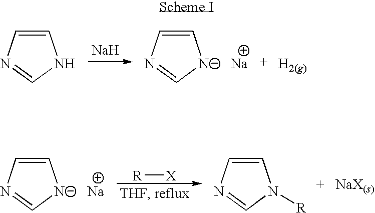

Producing a large quantity of ISS through the conventional method is relatively an expensive process. For example, as can be seen above, anhydrous solvents are required, and during the deprotonation reaction a sufficient quantity of solvent (typically>100 mL solvent per 10 g ISS produced) is required to maintain fluidity in the reaction vessel otherwise precipitated ISS limits reaction mixing, thereby impeding production of more ISS. While sodium hydride is relatively expensive, it does require safe handling and storage. The reaction also requires an inert atmosphere such as argon or nitrogen. These factors limit the quantity of ISS that can be produced by conventional methods. Furthermore, purification of ISS from this reaction also requires washing with more solvent and separation from any excess sodium hydride.

### Example 1

Imidazole (100.00 g, 1.47 mol) was melted at around 91° C. and NaOH pellets (55.868, 1.40 mol) were added while stirring. The disappearance of solid NaOH indicated reaction progress. One equivalent of water was produced during the reaction, and serves to hydrate ISS, while keeping the reaction fluid. ISS was isolated from unreacted imidazole by cooling the reaction and adding standard grade THF. Crystallized ISS was then filtered, washed with THF and dried in a vacuum oven over a drying agent such as CaSO₄ to produce dry ISS (93.068, 75% yield). Some materials were lost during collection, otherwise it is believed that the yield would be closer to 90%. ¹H NMR indicated only pure ISS remained after this process.

### Example 2

To a slurry mixture of ISS (5.00 g, 55.5 mmol) from Example 1 in a standard grade THF (50 mL) in a 100 mL round bottom flask was added 1-bromohexane (9.16 g, 55.5 mmol). The resulting mixture was heated overnight at reflux (65° C.) while stirring. The mixture was then cooled and the solids were removed by filtration. The resulting filtrate was concentrated, and the remaining yellow oil further concentrated under vacuum to produce the desired 1-hexylimidazole. ¹H NMR (not shown) confirmed the identity of the product to be 1-hexylimidazole (Yield=7.80 g, 92%).

### Example 3

ISS of Example 1 was also used to make other types of substituted imidazole compounds. For example, “gemini” or bis(imidazoles) was synthesized using the procedure described in Example 2 above but substituting a dihalide (e.g., 1,6-dibromohexane) for 1-bromohexane.

Bis(imidazoles) have found a wide variety of uses including units in coordination polymers, biological applications, photo-initiators, liquid crystals, and as difunctional monomers for step growth polymerizations. Furthermore, highly stable dicationic, gemini imidazolium salts such as those shown below:

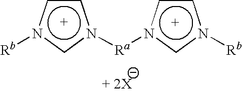

can be prepared from bis(imidazoles) as shown below:

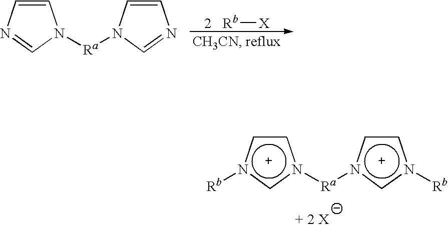

or through monoimidazoles as shown below:

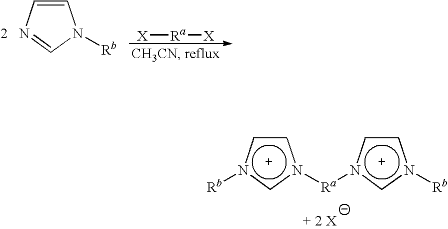

The nature of R^{b} groups gives rise to a number of different types of materials with a variety of possible applications. For example, gemini-imidazolium salts have been used as selective anion receptors, thermally stable lubricants, solvents for high temperature reactions and catalysts. If R^{b} groups are polymerizable, highly stable, crosslinked polymer electrolytes can be produced. When R^{b} groups are alkyl chains of at least 10 carbons, gemini lyotropic surfactants with ordered nanostructures can be produced around water or Room Temperature Ionic Liquids (RTILs).

### Example 4

N-substituted imidazoles (and similar compounds) currently find great utility as critical components of pharmaceuticals, antifungal and antibacterial agents, and corrosion inhibitors. In addition, there is great potential for their use as precursors to imidazolium-based room-temperature ionic liquids (RTILs). RTILs are salts that are molten at ambient conditions and feature desirable properties such as negligible vapor pressures, inflammability and thermal stability. There is great interest in these materials for a variety of engineering applications, such as liquid/vapor/gas separations, conductive fluids, lubricants, to name a few. Imidazolate salts are a critical component of the process to generate RTILs shown in general below:

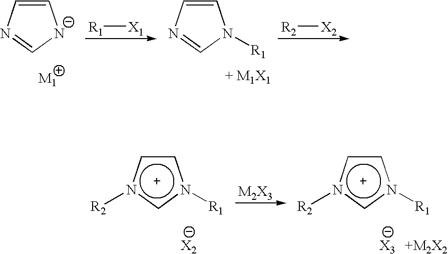

Generally, RTILs are produced when R₁ and R₂ are not equal and X₃ is a large, delocalized anion. A scheme for producing a widely studied and used RTIL, 1-ethyl-3-methyl imidazolium bis(trifluoromethane)sulfonamide is shown below:

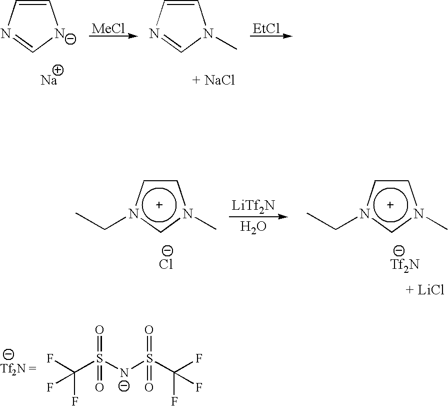

### Example 5

### Polymerization of bis(imidazoles) to Form poly(imidazolium) Salts

Tethered imidazoles or bis(imidazoles) is reacted with a difunctional compound to produce poly(imidazolium) salts.

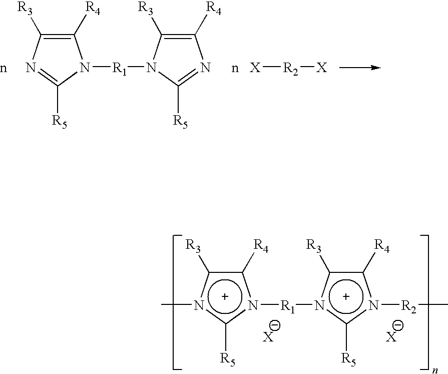

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. An isolated heteroaryl compound of at least 95% purity, wherein said heteroaryl compound is of the formula: 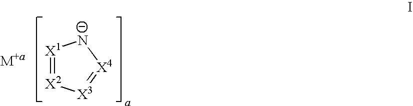
wherein
a is an oxidation state of M;
M is a metal, or R^{1a}R^{2a}R^{3a}R^{4a}N⁺, wherein each of R^{1a}, R^{2a}, R^{3a}, and R^{4a} is independently hydrogen or alkyl; and
each of X¹, X², X³, and X⁴ is independently N or CR⁵;
wherein
each R⁵ is independently hydrogen, halide, alkyl, heteroalkyl, aryl, aralkyl, cycloalkyl, (cycloalkyl)alkyl, heteroaryl, heteroaralkyl, heterocyclyl, or (heterocyclyl)alkyl;
or two adjacent R⁵'s along with the carbon atoms to which they are attached to form an optionally substituted aryl, heteroaryl, cyclycl, or heterocyclyl.

2. The isolated heteroaryl according to claim 1, wherein M is an alkali metal, an alkaline earth metal, or a transition metal.

3. The isolated heteroaryl according to claim 1, wherein a is 1 or 2.

4. The isolated heteroaryl according to claim 3, wherein a is 1.

5. The isolated heteroaryl according to claim 1 of the formula: 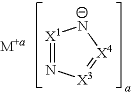
wherein a, M, X¹, X³ and X⁴ are those defined in claim 1.

6. The isolated heteroaryl according to claim 5, wherein X¹, X³ and X⁴ are CR⁵, wherein each R⁵ is independently that defined in claim 1.

7. The isolated heteroaryl according to claim 6, wherein R⁵ is H.

8. The isolated heteroaryl according to claim 7 of at least 98% purity.

9. A method for producing a heteroaryl compound of the formula: 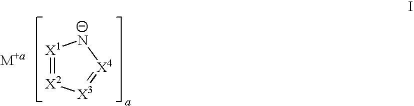
said method comprising:
reacting a compound of the formula: 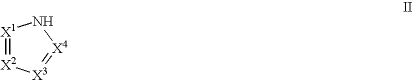
with a hydroxide compound of the formula:
M(OR)ₐ III
under conditions sufficient to produce the heteroaryl compound of Formula I,
wherein
a is an oxidation state of M;
each R is independently hydrogen, alkyl, cycloalkyl, or phenyl;
M is a metal, or R¹R²R³R⁴N⁺, wherein each of R¹, R², R³, and R⁴ is independently hydrogen or alkyl; and
each of X¹, X², X³, and X⁴ is independently N or CR⁵;
wherein
each R⁵ is independently hydrogen, halide, alkyl, heteroalkyl, aryl, aralkyl, cycloalkyl, (cycloalkyl)alkyl, heteroaryl, heteroaralkyl, heterocyclyl, or (heterocyclyl)alkyl;
or two adjacent R⁵'s along with the carbon atoms to which they are attached to form an optionally substituted aryl, heteroaryl, cyclyl, or heterocyclyl.

10. The method of claim 9, wherein the reaction is carried out under a reduced pressure.

11. The method of claim 9, wherein the reaction is carried out at a temperature near the melting point of Compound of Formula II.

12. The method of claim 9, wherein R is H.

13. The method of claim 12, wherein M is an alkaline metal, an alkaline-earth metal, or a transition metal.

14. The method of claim 9, wherein a is 1 or 2.

15. The method of claim 14, wherein a is 1.

16. The method of claim 9, wherein the reaction is carried out in an aqueous solution.

17. The method of claim 9, wherein the reaction is carried out in substantially a solvent free condition.

18. A method for producing a room-temperature ionic liquid (RTIL) compound of the formula: 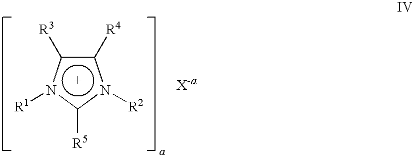
said method comprising
(i) reacting a compound of the formula: 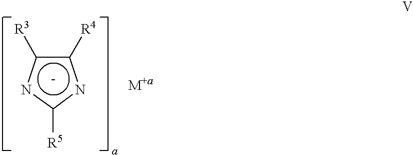
with a first reagent of the formula R¹-Z¹ under conditions sufficient to produce a mono-nitrogen substituted imidazole compound of the formula: 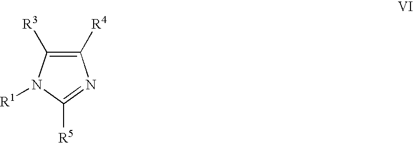
(ii) reacting compound of Formula VI with a second reagent of the formula R²-Z² under conditions sufficient to produce a RTIL compound of the formula: 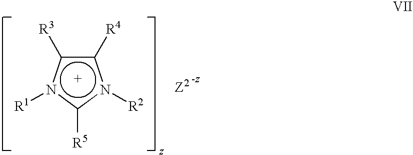
and
(iii) when Z² is different from X, then reacting compound of Formula VII with M¹ₐXₘ to produce the RTIL compound of Formula IV
wherein
a is an oxidation state of X;
m is an oxidation state of M¹;
z is an oxidation state of Z²;
X is a counter anion; and
each of R¹ and R² is independently alkyl, heteroalkyl, cycloalkyl, haloalkyl, silyl, siloxyl, aryl, alkenyl, or alkynyl;
each of R³, R⁴, and R⁵ is independently hydrogen, alkyl, cycloalkyl, heteroalkyl, haloalkyl, silyl, siloxyl, aryl, alkenyl, or alkynyl;
each of M and M¹ is independently a metal, or R^{1a}R^{2a}R^{3a}R^{4a}N⁺, wherein each of R^{1a}, R^{2a}, R^{3a}, and R^{4a} is independently hydrogen or alkyl; and
each of Z¹ and Z² is independently a leaving group.

19. The method of claim 18, wherein R¹ is alkyl, haloalkyl, or heteroalkyl.

20. The method of claim 18, wherein R² is alkyl, haloalkyl, or heteroalkyl.

21. The method of claim 18, wherein R³, R⁴, and R⁵ are hydrogen.
